(19) United States
(12) Patent Application Publication
(10) Pub. No.: US 2014/0242202 A1
(43) Pub. Date: Aug. 28, 2014

(54) Composition for Reducing Side- and After-Effects of Cancer Treatment

(71) Applicants: David Frankel, Kirkwood, MO (US); John Grearson, Brentwood, MO (US); Jay Locke, Harlingen, TX (US)

(72) Inventors: David Frankel, Kirkwood, MO (US); John Grearson, Brentwood, MO (US); Jay Locke, Harlingen, TX (US)

(21) Appl. No.: 14/255,329

(22) Filed: Apr. 17, 2014

Related U.S. Application Data
(62) Division of application No. 13/734,709, filed on Jan. 4, 2013.
(60) Provisional application No. 61/582,857, filed on Jan. 4, 2012.

(51) Int. Cl. A61K 38/17; A61K 36/28; A23L 1/29; A61K 36/9068

(52) U.S. Cl. CPC A61K 38/17; A61K 36/9068; A61K 36/28; A23L 1/296
USPC 424/756

(57) ABSTRACT

A compound for consumption by cancer patients, or others currently suffering from side effects such as those commonly associated with chemotherapy, that supplies herbal tonics with certain properties believed to ameliorate many of the side effects discussed above, and which has taste masking properties that improve the patient's tolerance of the supplement and likelihood to consistently consume the supplement. The compound is a mixture generally containing three core components: whey protein, which has a number of significant health benefits for cancer patients; hepatoprotectant and/or nephroprotectant agents(s), which reduce damage to the kidneys and liver; and anti-emetic agent(s), which reduce nausea and vomiting.

### CROSS REFERENCE TO RELATED APPLICATION(S)

This application is a Divisional of U.S. Utility patent application Ser. No. 13/734,709, filed Jan. 4, 2013, which in turn claims the benefit of U.S. Provisional Patent Application Ser. No. 61/582,857, filed Jan. 4, 2012. The entire disclosure of both the above documents is herein incorporated by reference.

### BACKGROUND

1. Field of the Invention

This disclosure is related to the field of nutritional supplements for patients suffering from malnourishment due to therapies, such as chemotherapy, used to treat diseases such as neoplastic disease, commonly known as tumors or cancers.

2. Description of the Related Art

The medical field is only just beginning to fully appreciate the role played by proper nutrition in treatment. Adequate nutritional support is shown to be critical to maintaining lean body mass, weight, and quality of life. Further, proper nutrition improves the tolerance and completion of treatment. This can be particularly true for patients undergoing treatments for cancer such as chemotherapy or radiation which are particularly grueling for the body.

Malnutrition is common in cancer patients and is recognized as an important component of adverse outcomes. Patients often experience various psychosocial difficulties when first diagnosed with cancer, and one of the common symptoms of these issues is that the patient experiences decreased appetite. Further, cancer patients commonly report early satiation, meaning the patient feels “full” after consuming fewer calories than usual. These two symptoms generally mean the patient experiences less overall caloric intake than when healthy, which in turn usually results in weight loss.

Cancer treatments and therapies often make the situation worse. For example, radiotherapy and chemotherapy patients commonly report taste alteration as a side effect, complaining that foods they once enjoyed taste metallic, bland, or rubbery while the patient undergoes treatment. Another well-known side-effect of cancer treatment is that the patient experiences nausea and vomiting. This in turn can cause the patient to experience taste aversion as the patient develops a negative psychological association of the flavors present in foods consumed just prior to treatment with the unpleasantness of nausea and vomiting. A similar mechanism can cause patients to develop texture aversions.

Thus, the patient, who is already under metabolic stress from suboptimal nourishment as a consequence of decreased appetite and early satiation, is even further disinclined to eat because of their perception that food tastes bad. Even worse, the treatment causes the patient to develop taste aversion to those foods that the patient does manage to consume, and it becomes increasingly difficult to find foods to which the patient has not yet developed a taste aversion. Thus, patients are commonly disinclined to maintain proper nutrition during the therapy.

Patients with food allergies may be at increased risk because the range of foods that they can safely tolerate is even smaller, and their diet is necessarily more restricted than that of a patient without food allergies. A number of conventional nutritional supplement materials can include more common allergens such as lactose, fructose, or fish products to provide their desired nutritional profiles for healthy individuals. Similarly, many commercially available nutritional supplements can include compounds that can cause drug interactions with compounds used as part of treatment regimens. Even after the patient completes the chemotherapy sessions, taste aversion can linger indefinitely, constricting the range of foods that the patient can tolerate. Still further, side-effects of cancer can also include glucose intolerance, insulin resistance, and increased lipolysis which can further worsen nutritional issues.

These factors result in various types and degrees of malnourishment. Malnourishment can inhibit recovery from cancer, inhibits the patient's ability to tolerate the treatments, which are hard on the body, and lowers the patient's enjoyment and quality of life. In effect, during the course of treatment it is generally very important for the person undergoing the treatment to remain “otherwise healthy.” Healthier individuals better tolerate the treatment's side-effects and keeps them motivated to succeed. At the same time, cancer progression and the treatment therapies work to discourage correct nourishment and thus the patient is effectively discouraged from remaining healthy by the act of treatment.

The most common form of malnutrition is known as protein-calorie malnutrition (“PCM”), which results from the inadequate intake of carbohydrate, protein, and fat to meet the body's metabolic requirements. For certain cancers, especially gastrointestinal cancers, the presence of tumors can reduce the body's ability to maintain fat stores and lean muscle tissue, which means that even if the patient appears to be eating enough food, the actual nutritional value that the body receives from that food is less than if the patient did not have tumors. As the body breaks down muscle tissue to generate the proteins it needs (and is not getting through diet), the patient generally losses lean body mass. Left untreated, PCM can lead to progressive wasting, weakness, and debilitation.

Another common nutritional disease affecting cancer patients is anorexia. While anorexia is commonly associated with anorexia nervosa, an eating disorder characterized by the obsessive fear of gaining weight, anorexia in cancer patients stems not from fear of food but from the loss of appetite and/or desire to eat. Anorexia is typically present in fifteen to twenty percent of all cancer patients at diagnosis, and may develop after diagnosis once treatment begins and the patient begins to experience the side effects described above. For individuals with widespread, advanced cancers, anorexia is a nearly universal side effect caused by physiological changes in metabolism that result from tumor growth. Anorexia is often exacerbated by the side effects of chemotherapy and radiotherapy mentioned above, such as taste and smell changes, nausea, vomiting, and taste aversion.

Shockingly, twenty to forty percent of deaths among cancer patients are believed caused not by the cancer itself, but by a progressive wasting syndrome known as cachexia, which develops in individuals who appear to be consuming adequate calories but experience inhibited maintenance of fat and lean muscle tissue due to the presence of tumors. As discussed, this disease is particularly prevalent among patients with gastrointestinal cancers. In effect, there is significant morbidity amongst those diagnosed with cancer not from the cancer itself, but from the malnutrition caused by side effects of both the disease and the treatment.

For patients in the advanced stages of cancer, the situation is even more precarious. As the body is placed under greater and greater stress by both the advancement of the disease, and the aggressiveness of the treatment, the body requires far greater demand for certain amino acids used to sustain normal blood sugar levels, which in turn are critical to supplying energy to the essential organs. If the patient's dietary intake of proteins does not supply sufficient quantities of these amino acids, the body acquires them by breaking down muscle tissue in a process known as catabolism. This can result in further damage to the body.

As mentioned, the consequences of insufficient protein in the diet include fatigue and reduced immune function, leading to greater susceptibility to disease, as well as delayed healing and declining ability to maintain normal life activities as a result of the gradual loss of lean muscle tissue and body mass. As the body consumes more and more muscle tissue to provide the needed amino acids, the mortality rate rises. As the patient's consumption of protein reserves approaches forty percent, the mortality rate approaches one hundred percent. Thus, if the body can obtain correct nutrition during treatment, a significant amount of morbidity in cancer patients can potentially be eliminated.

To summarize the above, cancer treatments are hard on the body and patients can die from the treatment itself. Where the patient survives and goes into remission after successful treatment, the body may be so damaged that the patient dies anyway or experiences severely decreased quality of life as the patient has lost muscle mass, is unable to undertake normal life activities, and has taste aversion to foods the patient previously found to be enjoyable. In some sense, these combined problems can result in patients, upon diagnosis, thinking they have a decreased chance of survival, and by that very belief, decreasing their chance of survival.

The scale of the impact of this problem is staggering. Approximately two a half million patients undergo chemotherapy each year, with more than a million of these patients beginning a chemotherapy regimen for the first time. More than forty percent (1.5 million) of chemotherapy patients experience suboptimal nutrition while undergoing chemotherapy, and twenty percent experience malnutrition, which, as mentioned, can result in death.

### SUMMARY

Because of these and other problems in the art, described herein are compounds for consumption by cancer patients that supply herbal tonics with certain properties believed to ameliorate many of the side effects of chemotherapy as discussed above, and which may include taste masking properties that improve the patient's tolerance of the supplement and likelihood to consistently consume the supplement. It is important to recognize that the compounds discussed herein are not designed to effect any form of cancer directly, or to interact directly with compounds used in existing chemotherapy or radiation regimens. Instead, these compounds are designed to provide for improved nutritional intake during the course of a separate treatment and that the compound used is designed to counteract some of the issues which can often lead to malnutrition.

There is described herein, among other things, a liquid composition comprising: whey protein; an anti-emetic; and a hepatoprotectant.

In an embodiment of the composition said anti-emetic provides the resultant taste profile of said composition and may comprise ginger and/or peppermint.

In an embodiment of the composition said hepaprotectant comprises milk thistle.

In an embodiment, the composition further comprises a taste masking agent such as, but not limited to, miraculin or a flavoring.

There is also described herein a liquid composition consisting essentially of: whey protein; an anti-emetic; and a hepatoprotectant.

In an embodiment of the composition said anti-emetic provides the resultant taste profile of said composition and may comprise ginger and/or peppermint.

In an embodiment of the composition said hepaprotectant comprises milk thistle.

There is also described herein a method of treating the side effects of cancer treatment comprising administering a liquid composition comprising the following: whey protein; an anti-emetic; and a hepatoprotectant.

In an embodiment of the method, the liquid composition is administered prior to a treatment being administered.

In an embodiment of the method the composition said anti-emetic provides the resultant taste profile of said composition and may comprise ginger and/or peppermint.

In an embodiment of the method the composition said hepaprotectant comprises milk thistle.

In an embodiment, of the method the composition further comprises a taste masking agent such as, but not limited to, miraculin or a flavoring.

### DESCRIPTION OF PREFERRED EMBODIMENT(S)

The compounds discussed herein are mixtures generally containing three core components although other components may be present and some of these core compounds are not present in further alternative embodiments. Firstly, the compound includes a nutritional source. This provides readily accessible nutrients and particularly proteins for the person consuming the compound. In an embodiment, the nutritional course comprises whey protein, which has a number of significant health benefits for cancer patients and is a well-known and understood nutritional source, particularly for proteins. Secondly, the compound includes a hepatoprotectant and/or nephroprotectant agents(s), which is intended to reduce damage to the kidneys and/or liver. Thirdly, the compound includes anti-emetic agent(s), which reduce nausea and vomiting.

Whey protein comes from milk serum (also known as whey) which is a liquid byproduct of cheese and casein production left over after milk has been curdled and strained. Whey protein consists of globular proteins isolated from whey, typically in a mixture of approximately 65% β-lactoglobulin and 25% α-lactalbumin, as well as 8% serum albumin. Whey protein is believed to contain properties conducive to cancer-fighting and tolerance of treatment. Firstly, whey protein is rich in essential amino acids. Essential amino acids are those amino acids which are not generated in the body, and which must be acquired through diet. Because of the problems in the art described above, cancer patients undergoing treatment frequently experience breakdown of muscle tissue to supply critical proteins and amino acids that the body should be (but is not) receiving through diet. By consuming whey protein, the patient should acquire sufficient essential amino acids to mitigate or negate this problem in the art. This in turn causes the patient to feel healthier and more normal, and to experience increased quality of life, decreased mortality, and to better tolerate treatment.

Whey protein is an ideal source of branch chained amino acids, which are believed to stimulate protein synthesis, though the precise operational pathway of this effect is not commonly understood. Because of the problems in the art described above, cancer patients undergoing treatment frequently experience breakdown of muscle tissue to supply critical proteins and amino acids that the body should be (but is not) receiving through diet. By consuming whey protein, the patient can increase protein synthesis, mitigating or eliminating net muscle tissue lost to catabolism. Again, this in turn causes the patient to feel healthier and more normal, and to experience increased quality of life, decreased mortality, and to better tolerate treatment.

Whey proteins also facilitate gastrointestinal tolerance by emptying the stomach of food and reducing the potential for vomiting. Because of problems described above in the art, cancer patients may experience nausea and vomiting as a result of certain cancer treatments, leading to taste aversion. By consuming whey protein, the patient can reduce vomiting by simply having less to vomit, which can limit taste aversion, improve the patient's tolerance of food, and mitigate malnourishment due to loss of food which has been consumed but not fully processed. Again, this in turn causes the patient to feel healthier and more normal, and to experience increased quality of life, decreased mortality, and to better tolerate treatment.

Whey proteins are also believed to promote anabolism, growth, healing and recovery, and have anti-inflammatory properties. By consuming whey protein, cancer patients should be able to hasten healing and shorten recovery time from various cancer treatments, including radiotherapy and chemotherapy treatments, as well as surgical procedures to remove tumors.

Whey proteins, in purest form, contain little or no fat, lactose or cholesterol, and are gluten-free. This means that whey proteins may be safely consumed even by many patients with common food allergies and will generally not provide adverse materials which could lead to complications or drug interactions.

Although these properties make whey protein an ideal source of nutritional support for individuals under metabolic stress such as cancer patients, there remains the problem of compliance to provide whey proteins. Whey proteins can provide these benefits to patients only when consumed and one of the problems in the art is that cancer patients are disinclined to consume even the foods they enjoy. Cancer patients are even less likely to consume whey protein, which is generally perceived as having an unpleasant taste profile (often a bitter milk profile), and which has an even worse taste profile to patients undergoing cancer treatments and experiencing taste alteration. As a major issue, whey protein is often most delectable when mixed into milk. However, milk can be hard to digest and can result in increased concern for nausea. Further, those who are lactose intolerant can generally not consume whey protein in milk, forcing the user in that case to consume the product with a less desirable taste profile.

In the absence of dysphagia, the taste profile problem cannot be avoided by merely crushing these compounds into the form of a capsule that can be swallowed to bypass taste receptors. However, whey proteins are often bulky and difficult to confine to a form (such as a capsule) that can bypass the taste receptors. If taken in capsule form, the patient would be required to consume a very large number of capsules, increasing the risk of choking and damage to the throat and esophagus. Second, capsules absorb more slowly than do liquids, meaning that the time for the component materials to reach potency in the body is increased. As described below, the supplement is more effective if consumed just prior to treatment, and rapid absorption of the component materials is therefore desirable.

Thus, patients must consume whey proteins in a form which must be tasted. Specifically an oral medication because the desired properties operate most effectively when consumed as a tonic. Because a tonic, unlike a pill, will necessarily contact the taste buds, the patient will taste the tonic and the unpleasant taste profile will increase patient non-compliance with consuming the nutritional supplement treatment.

To solve this problem, in an embodiment, Applicants' compound includes a taste masking agent. By way of example and not limitation, one form of this taste masking agent comes from the plant Zingiber officinale, whose rhizome is commonly known as ginger. Ginger is a well-known flavoring agent in the culinary arts, with a strong and generally pleasant taste which reduces the unpleasant flavors of whey protein. Ginger is a particularly useful flavoring agent because it doubles as an anti-emetic due to it including 5-HT₃ receptor antagonists, reducing the nausea and vomiting that also typically follows a chemotherapy session. Along with whey protein, which stimulates stomach emptying, the anti-emetic properties of ginger address the problems in the art described above by further reducing feelings of nausea. This can result in improved caloric intake, reduced malnourishment, and thus reduced loss in muscle tissue and improved overall quality of life for the patient.

While ginger is a particularly useful anti-emetic because it has a positive flavor profile, the compound need not utilize it as the only, or even as one, anti-emetic. In alternative embodiments, other anti-emetics with strong generally pleasant flavor profiles such as, but not limited to, Ajwain and peppermint may alternatively or additional be used. Further, these types of flavorful anti-emetics may be combined with, or replaced by, other anti-emetics such as, but not limited to, 5-HT₃ receptor antagonists, antihistimines, and cannabinoids (including marijuana) where the taste profile of these may not be used or may be concealed by flavoring agents such as is discussed below.

In an embodiment, it is desirable to not only provide the nutritional supplementation, but to further provide additional compounds with protectant properties that serve to directly protect various body systems from the potentially dangerous effects of cancer treatment. Some known adverse side effects include damage to the endocrine system to specific organs, including the kidneys and liver. By further providing in the mixture compounds with hepatoprotectant and nephroprotectant properties, vulnerable organs such as the liver and kidneys may better tolerate cancer treatment.

By way of example and not limitation, one such compound with hepatoprotectant and nephroprotectant properties is the plant silybum marianum, commonly known as milk thistle, which is believed to have both hepatoprotectant and nephroprotectant properties. However, milk thistle also is generally perceived to have an unpleasant, bitter taste. As with whey protein, patients undergoing cancer treatment are already disinclined to consume even their favorite foods, nonetheless unpalatable flavors such as milk thistle. Further, the bitter flavor of milk thistle can result in a product which is even more bitter tasting. As with whey protein, the taste masking properties of ginger can reduce the unpleasant taste profile of milk thistle, improving the likelihood that the patient consumes the supplement and receives the benefits of the whey protein, milk thistle, and ginger.

Other additives with protectant properties may also be included in the supplement which reduce or eliminate other negative side effects of common cancer treatments. By way of example and not limitation, such additional protectants may include, individually or in combination, materials to resist damage to and loss of hair and related tissue, and materials to resist damage to and loss of tooth and gum tissue (e.g. fluorides). Protectants may also be included to resist xerostomia (more commonly called “dry mouth” or a lack of saliva) or resist mucositis, the inflammation and ulceration of the mucous membranes lining the digestive tract. Mucositis and specifically oral mucositis (which refers to the particular inflammation and ulceration that occurs in the mouth) are common and often debilitating complications of cancer treatment.

Although ginger is a preferred initial taste masking agent because of its secondary anti-emetic properties, Applicants contemplate that the compound may also include other natural and artificial flavoring compounds, giving the supplement the taste of, for example, vanilla, strawberry, or chocolate. These additional taste masking compounds may (but need not) also provide desirable cancer-fighting properties and/or other nutritional value.

One such additional flavoring agent are sweetening proteins. These often have the side effect of taste alteration. One such protein is a glycoprotein known as miraculin, one source of which is the plant synsepalum dulcificum, colloquially known as the “miracle berry.” Miraculin, when in contact with human sensory receptors for taste, is believed to alter the brain's perception of taste. For example, acidic flavors are perceived as sweet while miraculin is bound to the tongue's taste receptors. However, miraculin does not alter the chemical properties of food. Miraculin can be added to the tonics described herein to further provide taste masking without compromising the desirable chemical properties of the mixture. Other similar materials include curculin and cynarin. One concern is the effect that miraculin (or related agents) has on primarily bitter materials since, as discussed above, an embodiment of the present compound has a generally bitter taste. In some cases, mirculin does appear to sweeten the taste of bitter agents, while other people appear to notice no change. In one embodiment, the bitter taste is masked by including a particularly sour taste (which miraculin is generally accepted as sweeting) such as, but not limited to, ascorbic acid or more natural flavors such as lemon or lime juice as a mask taste that can overpower the bitter taste of the other ingredients.

Further, because miraculin causes the patient to experience a taste profile that the supplement does not truly possess, if the patient develops a taste aversion, the aversion will be to a flavor that generally does not actually exist other than when the specific tonic is formed and contains miraculin. That is, the patient consuming a nutritional supplement with a particular flavoring (e.g. a strong lemon ginger flavoring to use an example) that contains miraculin is less likely to develop a taste aversion to that particular flavoring because miraculin alters the patient's perception of a flavor that is likely uncommon in the culinary arts anyway. Thus, if the patient develops an aversion, it will be to the patient's altered perception of an uncommon flavor which may reduce their aversion to a non-altered profile. When the patient completes treatment and encounters that flavoring without miraculin, the patient's aversion is less likely to cause a negative reaction to that flavoring, and the patient can enjoy those foods normally.

In addition to these modifications, the mixture can also be modified with other compounds that have properties which aid in cancer-fighting, treatment tolerance, or overall nourishment. For example, generally “healthy” oils, such as certain plant oils, can be included as a source of fats.

By way of example and not limitation, the mixture could include arginine, an non-essential amino acid produced in some quantity by the human body, but generally insufficient to meet the body's total need, such that at least some dietary supplementation is required to prevent adverse impacts to health. Arginine is believed to have cancer-fighting and treatment-tolerating properties such as improved healing, immune function, hormonal regulation and protein synthesis.

By way of example and not limitation, the mixture could also, or alternatively, include glutamine, a non-essential amino acid produced in abundance within the body, and which is believed to have cancer-fighting and treatment-tolerating properties such as improved healing and tolerance of illnesses and injuries such as burns and other side effects of cancer treatment.

By way of example and not limitation, the mixture could include a vitamin and mineral blend formulated to provide other nutrients, alone or in combination, as needed by the patient. Such vitamins and minerals include but are not limited to: potassium citrate, dicalcium phosphate, dendritic salt, trimagnesium phosphate, potassium chloride fcc, maltodextrin, choline bitartrate, Vitamin C USP, zinc gluconate, ferric orthophosphate, Vitamin E USP, niacinamide USP, copper gluconate, d-calcium pantothenate USP, manganese sulfate, Vitamin A USP, Vitamin B6 USP, Vitamin B12 USP, Vitamin D3 USP, and Vitamin B12 USP.

In addition to taste-masking, various other non-essential compounds may be added to the mixture, individually or in combination. By way of example and not limitation, creamers, thickening agents, fibers, suspensions, and other additives may be added to alter and improve the texture of the supplement for purpose of avoiding texture aversion, or merely to improve the overall palatability of the supplement. By way of example and not limitation, one such example of an additive is a creamer consisting of partially hydrogenated soybean oil, corn syrup solids, sodium caseinate, mono- and diglycerides, sodium citrate, salt, dipotassium phosphate, an anti-caking agent, carrageenan, and artificial flavor(s).

Other additives may be included in the mixture for various other purposes. By way of example and not limitation, such other additives include preservatives to preserve the supplement in shipping and to provide adequate shelf-life for the supplement.

In addition to the materials which may be optionally included as part of a composition, the compositions can also be designed for what they do not include. As an example, it is preferable that the compositions not include lactose, gluten, or fructose which are common food allergens. Similarly, avoiding soy-, nut-, or fish-sourced ingredients can be beneficial. In addition to known or common allergens, a bigger issue is that it is preferable that the composition not include compounds that can create known drug interactions with drugs that are commonly used in the treatment of cancer. For example, many cancer patients have surgery and may be taking anti-coagulants related to that surgery. It is therefore desirable that the composition avoid including materials that may cause an adverse reaction or are also anti-coagulants. One such example being Vitamin K. Similarly, stimulants (such as caffeine) may be harmful for a patient and should not be included.

In application, the mixture is intended for use in stressed patients under circumstances where inadequate nourishment or malnourishment may arise. Use is particularly contemplated in circumstances where inadequate nourishment or malnourishment is caused or contributed to by reduced appetite or disinclination to eat as a consequence of nausea, frequent regurgitation, and/or taste aversion. Because the compound is specifically formulated to promote healing and provide protection to major digestion organs, notably the liver and kidneys, the compound is particularly applicable to patients undergoing radiotherapy and chemotherapy treatment.

In application, the mixture is primarily intended to be consumed in close proximity to treatment, preferably before treatment, such that the flavor of the supplement is the last flavor that the patient consumes before experiencing the nausea and/or vomiting commonly associated with cancer treatments such as radiotherapy and chemotherapy. Thus, if the patient develops a taste aversion, it likely will be to the flavor of the supplement, and not any other food consumed prior to treatment, because the flavor of the supplement is the last flavor the patient experienced prior to becoming ill. Further, this can allow for the compound to be present in the body during and immediately after the therapy which may improve the anti-nausea effects.

As described above, the taste of even the flavored supplements (e.g., chocolate, vanilla) is uncommon in the culinary arts due to the presence and natural flavor of other components such as whey protein and milk thistle, and any taste aversion the patient develops to this uncommon flavor is less likely to impact the patient's ability to enjoy food after treatment. In the case of a supplement which includes miraculin, the taste aversion will be to the altered uncommon flavor of the supplement, which is even one step further removed from flavors generally found in the culinary arts.

Further, consuming the supplement in close proximity to treatment maximizes the body's use of materials in the supplement with protectant properties, such as milk thistle, because the potency of those compounds in the body, and thus the body's use of the protectants and other desirable characteristics to resist the side effects of treatment, will taper off over time.

The mixture is preferably provided as a liquid tonic, or as a solid which is suspended or dissolved in a liquid (such as, for example, a powdered form) prior to consumption. In order to form the product as a liquid, liquid bases such as, but not limited to, water may be used to provide bulk or suspend the components. As discussed above, the liquid form is preferable because of the inherent bulk of components such as whey protein and milk thistle, which cannot be condensed or concentrated into a small capsule which can be swallowed in manageable quantities. Further, because the supplement should be taken just prior to treatment so that the supplement may prevent taste aversion, the supplement should be in a form which allows for rapid absorption of the component materials, and liquids are known to provide more rapid absorption than solids or capsules.

In an embodiment, the composition is formed as a dry powder having a vanilla flavor for suspension in a liquid such as, but not limited to, water and includes whey protein isolate, cane sugar, high oleic sunflower oil, modified food starch, maltodextrin, natural flavors, resistant maltodextrin, medium chain triglycerides, potassium citrate, xanthan gum, sunflower lecithin, ginger root extract, dicalcium phosphate, tricalcium phosphate, dipotassium phosphate, milk thistle extract, magnesium phosphate, choline bitartrate, potassium chloride, sodium chloride, stevia (reb a), zinc gluconate, ascorbic acid, ferric orthophosphate, dl-alpha-tocopheryl acetate, niacinamide, copper gluconate, calcium d-pantothenate, manganese sulfate, pyridoxine hydrochloride, riboflavin, thiamin hydrochloride, vitamin a palmitate, chromium picolinate, folic acid, biotin, sodium molybdate, potassium iodide, sodium selenite, cholecalciferol, and cyanocobalamin. In a still further embodiment, these ingredients comprise all the ingredients of the dry powder.

In another embodiment, the composition is formed as a dry powder having a chocolate (cocoa) flavor for suspension in a liquid and includes whey protein isolate, cane sugar, high oleic sunflower oil, modified food starch, maltodextrin, cocoa (processed with alkali), natural flavors, resistant maltodextrin, medium chain triglycerides, potassium citrate, xanthan gum, sunflower lecithin, ginger root extract, dicalcium phosphate, tricalcium phosphate, dipotassium phosphate, milk thistle extract, magnesium phosphate, choline bitartrate, potassium chloride, sodium chloride, stevia (reb a), zinc gluconate, ascorbic acid, ferric orthophosphate, dl-alpha-tocopheryl acetate, niacinamide, copper gluconate, calcium d-pantothenate, manganese sulfate, pyridoxine hydrochloride, riboflavin, thiamin hydrochloride, vitamin a palmitate, chromium picolinate, folic acid, biotin, sodium molybdate, potassium iodide, sodium selenite, cholecalciferol, and cyanocobalamin. In a still further embodiment, these ingredients comprise all the ingredients of the dry powder.

The disclosed composition addresses several important problems associated with the treatment of neoplastic disease. It provides critical caloric intake to maintain body mass, improves muscle tissue retention, and supplies the body with amino acids to hasten healing and improve immune function, and provides nephroprotectants and heptaprotectants.

The disclosed composition also addresses a problem that continues to afflict cancer patients long after the treatment regimen has concluded: taste aversion. By altering the patient's perceived taste of the supplement, the patient's taste aversion, if any, will be to a flavor that does not actually exist because the actual flavor of the nutritional supplement has been altered, in the patient's perception, by miraculin.

While the invention has been disclosed in connection with certain preferred embodiments, this should not be taken as a limitation to all of the provided details. Modifications and variations of the described embodiments may be made without departing from the spirit and scope of the invention, and other embodiments should be understood to be encompassed in the present disclosure as would be understood by those of ordinary skill in the art.

1. A method of treating the side effects of cancer treatment comprising administering a liquid composition comprising the following:
whey protein;
an anti-emetic; and
a hepatoprotectant.

2. The method of claim 1 wherein said liquid composition is administered prior to a treatment being administered.

3. The method of claim 1 wherein said anti-emetic provides the resultant taste profile of said composition.

4. The method of claim 1 wherein said anti-emetic comprises ginger.

5. The method of claim 1 wherein said hepaprotectant comprises milk thistle.

6. The method of claim 1 wherein said liquid composition further comprises a taste masking agent.

7. The method of claim 6 wherein said taste masking agent comprises miraculin

8. The method of claim 6 wherein the taste masking agent comprises a flavouring.
